(19) United States
(12) Patent Application Publication
(10) Pub. No.: US 2019/0298296 A1
(43) Pub. Date: Oct. 3, 2019

(54) Disposable Stethoscope Cover

(71) Applicant: Erin E. Boatsman, La Mesa, CA (US)

(72) Inventor: Erin E. Boatsman, La Mesa, CA (US)

(21) Appl. No.: 15/846,503

(22) Filed: Dec. 19, 2017

Related U.S. Application Data
(60) Provisional application No. 62/439,939, filed on Dec. 29, 2016.

(51) Int. Cl. A61B 7/02

(52) U.S. Cl. CPC A61B 7/02; A61B 46/10

(57) ABSTRACT

The device of the present invention is a disposable stethoscope cover for an acoustic stethoscope having a single-sided or dual-sided chest piece with two diaphragm ends or a diaphragm end and a bell end. The device has a semi-rigid single piece or multiple piece cover having semi-rigid snap-fit or pressure fit cover elements that fit over the diaphragm and/or bell ends of the stethoscope. In a two sided configuration of the device it has a first and second ends connected by a tether flap. The diaphragm cover elements have an ascending skirt about a majority of said perimeter edge for receiving and securing the semi-rigid flat surface cover element parallel to the diaphragm of the stethoscope and a U-shaped circular ring end to be received and secured to the bell end of the stethoscope.

### CROSS-REFERENCE TO RELATED APPLICATIONS

Not Applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates generally to a protective and disposable cover for an acoustic stethoscope. More specifically, a disposable cover for a diaphragm end and/or a bell end of a medical stethoscope that helps prevent the transmission of undesirable agents or pathogens from one patient to another when a physician conducts multiple examinations in which a stethoscope is utilized.

### BACKGROUND OF THE INVENTION

The practice of medicine, by its very nature, is best performed in a sterile environment. In this regard, the devices used by a physician during an examination should be sterilized after being used on a patient and before use on a subsequent patient. Some of these devices such as an Otoscope, a device used for visual examination of the eardrum and ear passage, have disposable specula which is the part of the scope that is inserted into the ear during examination. Similarly, a variety of disposable devices have been developed for the stethoscope. U.S. Pat. Nos. 7,424,929 and 7,775,807 to Martinez discloses a sanitary stethoscope cover comprising a sanitized disk encased by an expandable binding ring that maintains the disk and allows for attachment of the device to a diaphragm or bell of a stethoscope. Unfortunately, many stethoscopes used by medical doctors are dual sided usually having both diaphragm and bell ends. Consequently, both ends should be capped prior to use. This means that two covers must be affixed one on each end of the stethoscope after each examination. In addition, because they are secured by the elastic property of the binding ring, both hands are required to affix each cover to the stethoscope. In addition, two covers must be utilized for each examination. Further, attachment of the covers can be cumbersome and time consuming.

U.S. patent application no. 2016/0199021 to Dula discloses a stethoscope cover having a flexible disc shaped body made of a sanitary material and an elastic band secured along its circumference that is able to encase the entire chest piece or just one end of the stethoscope. Unfortunately, acoustic stethoscopes are designed to enable a physician to hear the internal sounds created by the body. In this regard, a semi-rigid acoustic membrane is used to accurately transmit those sounds. The flexible sanitary material described in Dula could interfere with the transmission of those sounds. In addition, securing the device to a stethoscope can be cumbersome and time consuming.

Consequently, there is a need for a disposable stethoscope cover that is provided in a single piece construction or a multiple piece construction that does not require the use of both hands to affix to a stethoscope. Thereby eliminating the cumbersome and time consuming aspects of using previous stethoscope covers.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

In one aspect of the present invention a device is disclosed and described herein that provides a stethoscope cover for an acoustic stethoscope with a dual-sided chest piece having a diaphragm end and a bell end. The device has a semi-rigid single piece cover having a first end that affixes to the diaphragm end and a second end that affixes to the bell end wherein both ends are connected by a tether flap, the first end having a flat surface and a perimeter edge. The perimeter edge having an ascending skirt about a majority of the perimeter edge that receives the first end securing the flat surface of the first end parallel to the diaphragm of the stethoscope. The second end having a U-shaped circular ring able to be secured to the bell end.

A second aspect of the present invention is a stethoscope cover for an acoustic stethoscope with a dual-sided chest piece having a first diaphragm end and a second diaphragm end. In this configuration the device having a semi-rigid single piece cover with a first end that affixes to the first diaphragm end and a second end that affixes to the second diaphragm end wherein the first and second ends are connected by a tether flap. The first end having a first flat surface and a first perimeter edge, wherein the first perimeter edge having a first ascending skirt about a majority of the first perimeter edge that is able to secure the first flat surface of the first end parallel to the first diaphragm of said stethoscope. The second end having a second flat surface and a second perimeter edge, wherein the second perimeter edge having a second ascending skirt about a majority of the second perimeter edge able to secure the second flat surface of the second end parallel to the second diaphragm of the stethoscope.

A third aspect of the present invention is a stethoscope cover for an acoustic stethoscope with a dual-sided chest piece having a first diaphragm end and a second diaphragm end. The device having a first semi-rigid circular cover to be received by the first diaphragm end, a second semi-rigid circular cover to be received by the second diaphragm end and a flexible connector having a first end to which the first semi-rigid circular cover is affixed and a second end to which the second semi-rigid circular cover is affixed, wherein the first and second flexible connectors are connected by a tether flap. The flexible connector first end having a perimeter edge with an ascending skirt about a majority of the perimeter edge, wherein the ascending skirt receives said first diaphragm end securing the first semi-rigid circular cover parallel to the first diaphragm of said stethoscope. The flexible connector second end having a perimeter edge with an ascending skirt about a majority of the perimeter edge for securing the second semi-rigid circular cover parallel to the second diaphragm of the stethoscope.

A fourth aspect of the present invention is a stethoscope cover for an acoustic stethoscope with a dual-sided chest piece having a diaphragm end and a bell end. The device having a semi-rigid circular cover and a flexible connector having a first end to which the semi-rigid circular cover is affixed and a second end for receiving the bell end wherein the first and second ends are connected by a tether flap. The first end having a perimeter edge with an ascending skirt about a majority of the perimeter edge to receive the diaphragm end securing the first semi-rigid circular cover parallel to the diaphragm of the stethoscope. The second end having a U-shaped circular ring able to receive said bell end.

A fifth aspect of the present invention is a stethoscope cover for an acoustic stethoscope having at least one diaphragm end. The device having a semi-rigid single piece cover that affixes to the diaphragm end. The single piece having a flat surface and a perimeter edge with an ascending skirt to receive the diaphragm end securing the flat surface parallel to the diaphragm end of the stethoscope.

A sixth aspect of the present invention is a stethoscope cover for an acoustic stethoscope having at least one bell end. The device having a semi-rigid single piece cover that affixes to the bell end, wherein said semi-rigid single piece cover having a U-shaped circular ring able to receive the bell end.

In one embodiment of each aspect of the invention, the cover is disposable. In another embodiment of each aspect, the device further comprises a removal means such as a tab.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other configurations for carrying out the several purposes of the present disclosed device. It is important, therefore, that the claims be regarded as including such equivalent construction insofar as they do not depart from the spirit and scope of the present invention.

The objects, features, and advantages of the invention will be brought out in the following part of the specification, wherein detailed description is for the purpose of fully disclosing the invention without placing limitations thereon.

### BRIEF DESCRIPTION OF THE DRAWINGS

FIG. 1 shows one embodiment of the single piece configuration of the device of the present invention for a stethoscope having a dual sided chest piece with one diaphragm end and one bell end.

FIG. 2 shows one embodiment of the multiple piece configuration of the device of the present invention for a stethoscope having a dual sided chest piece with one diaphragm end and one bell end.

FIG. 3 shows a perspective view of the multiple piece configuration of FIG. 2 without the tab or flap.

FIG. 4 shows a side profile view of the multiple piece configuration of FIG. 2.

FIG. 5 shows a top side profile of the multiple piece configuration of FIG. 2.

FIG. 6 shows a bottom side profile of the multiple piece configuration of FIG. 2.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

### Definitions

The term “affixes” as used herein refers to a removable interaction of the stethoscope diaphragm end or bell end with the semi-rigid single piece or multiple piece cover having compressible edges that grip the stethoscope diaphragm end or bell end along their perimeter edges by pressure fit.

The term “connected” as used herein with respect to the first and second ends of the disposable stethoscope cover, refers to an element that is either integral to or separate from the elements that fit over the stethoscope diaphragm(s) and/or bell and join these elements together so that they are used as a single stethoscope cover. In many of the embodiments of the present invention, the term “connected” refers to the function of the tether flap.

### Disposable Cover

In one aspect, the present invention is a cover 10, 20 for a dual sided acoustic stethoscope having at least one diaphragm end 14, 36 or at least one diaphragm end 14, 36 and one bell end 16, 38. The stethoscope cover 10, 20 is prepared from a piece of polymer and comprises a semi-rigid single piece cover 12, 32 having a first end 14, 36 that affixes to the diaphragm end and a second end 16, 38 that affixes to the bell end or a second diaphragm of a dual sided stethoscope. The first 14, 36 and second ends 16, 38 are connected by a tether flap 18, 42. The first end 14, 36 having a flat surface and a perimeter edge, wherein the perimeter edge contains an ascending skirt 22, 44 about a majority of the perimeter edge for affixing to the edge of the stethoscope diaphragm end. The ascending skirt 22, 44 secures the flat surface of the first end parallel to the diaphragm of the stethoscope. The second end 16, 38 may have a U-shaped circular ring to receive the bell end. The U-shaped circular ring may encompass a majority of the bell end. Correspondingly, if the second end 16, 38 of the dual sided stethoscope is a second diaphragm, then the second end of the cover will also have a flat surface and a perimeter edge with an ascending skirt about a majority of the perimeter edge for affixing to the edge of the stethoscope's second diaphragm end such that the cover is parallel to the diaphragm.

The stethoscope cover of the present invention 10, 20 may be provided in a variety of configurations. These configurations include: a single piece form molded cover 10 comprising two snap fit cover pieces 14, 16 one for each end of a dual sided stethoscope having an ascending skirt 22 along its edge for receiving the diaphragm end of the stethoscope connected via a tether 18, the dual sided stethoscope may have two diaphragm ends or one diaphragm end 14 and a bell end 16; a multiple piece cover 20 comprising one or two form molded cover pieces one for each end of a dual sided stethoscope having an ascending skirt 44 along its edge for receiving the diaphragm end of the stethoscope connected to a form molded tether 42 that may also provide the snap or elastic fit elements for connecting the covers to both ends of the dual sided stethoscope, the dual sided stethoscope may have two diaphragm ends or one diaphragm end 36 and a bell end 38; a multiple piece cover comprising two form molded cover pieces connected to a form molded tether 42 that may also provide the snap or elastic fit element for connecting the covers to both diaphragms of a dual diaphragm stethoscope; a multiple piece cover comprising one form molded cover piece 32 connected to a form molded tether 42 that may also provide the snap or elastic fit element for connecting the cover to the diaphragm end of a dual sided stethoscope, the other end of the tether 42 being form molded to cover the bell end 38; a single piece form molded cover comprising a single snap fit cover piece for a dual sided or single sided stethoscope to cover the diaphragm end of stethoscope; and a single piece form molded cover comprising a single snap fit cover piece for a dual sided or single sided stethoscope to cover the bell end of stethoscope. Any of the device configurations may further comprise a removal means 24, 46 on one or both of the first 14, 36 and/or second 16, 38 ends. In one embodiment, this removal means 24, 46 is a tab or flap extending from the edge of the diaphragm and/or bell ends of the device.

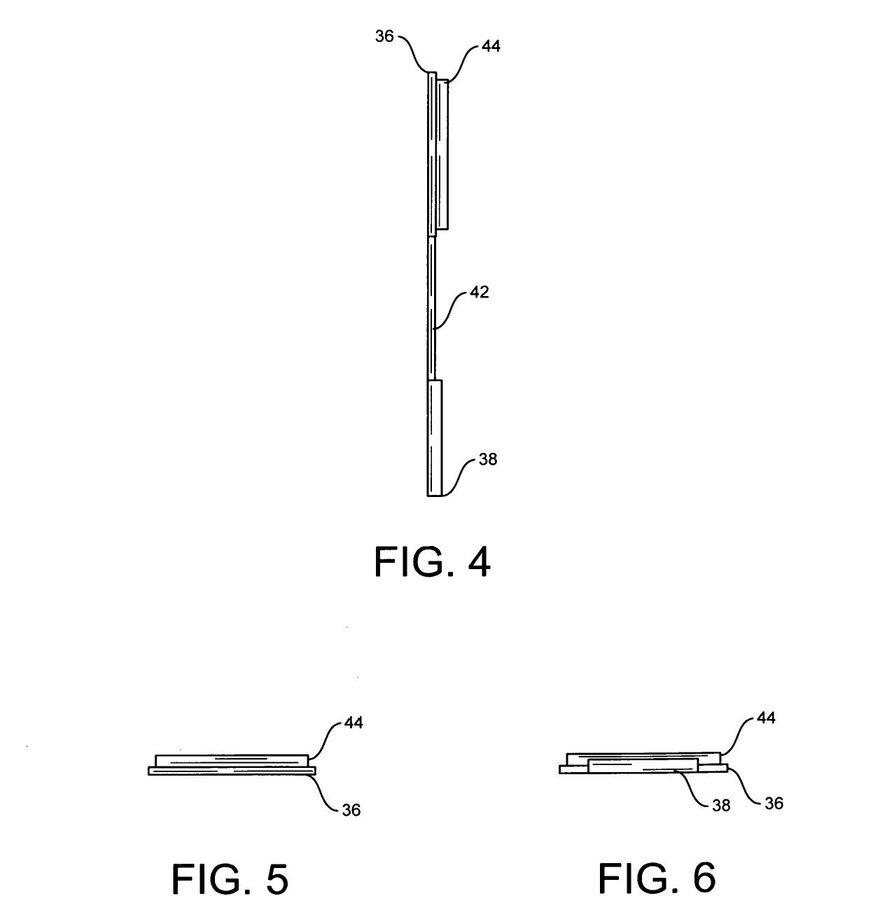

In the simplest of these configurations, the device 20 is prepared in two pieces for a dual sided stethoscope having one diaphragm end 36 and one bell end 38 (see FIGS. 2 and 3-6). The two pieces include a semi-flexible polymer connector 34 having a tether 42 with a first end 36 being a relatively circular loop for inserting the semi-rigid cover that fits over the diaphragm end of the stethoscope and a second end 38 with a U-shaped circular ring to receive the bell end. The circular loop of the first end 38 having a groove along its interior diameter for receiving a ridge provided in the perimeter edge of the semi rigid cover 32 and the semi-rigid cover 32 having an ascending skirt 44 along its edge for receiving the diaphragm end of the stethoscope.

In the next simplest configuration the device is prepared for a dual sided stethoscope having two diaphragm ends. The three pieces include a tether having two relatively circular loops, one on either end, that are each able to receive a semi-rigid cover. The circular loop of the second end having a groove along its interior diameter for receiving a ridge provided in the perimeter edge of the semi rigid cover and the semi-rigid cover having an ascending skirt along its edge for receiving the diaphragm end of the stethoscope. Each of these covers are appropriately sized to fit over the corresponding diaphragm of the stethoscope being used.

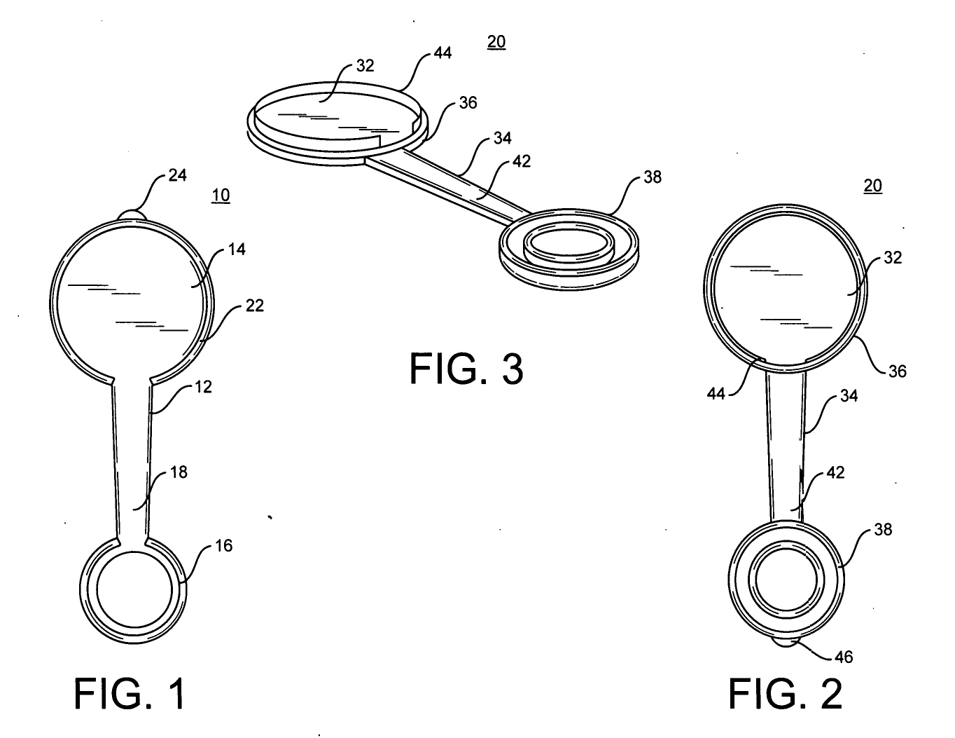

The single piece stethoscope cover 10 (see FIG. 1) may be made of a variety of polyolefin polymers including polyethylene, polypropylene, polyvinyl chloride and copolymers thereof. In a multiple piece configuration, a diaphragm cover 12 may be made of the polyolefin polymers listed above, while the flexible connector 34 is made of a more flexible elastic polymer in which the diaphragm covers 32 may be mounted. Mounting may be performed by a variety of methods know in the art. The diaphragm covers may be simply held within the relatively circular loop by the elastic force of the loop contracting about the perimeter edge of the diaphragm cover 32, a flexible adhesive may be utilized to secure the diaphragm cover 32 within the flexible tether loop, or the diaphragm cover 32 may be fused by heat into the tether loop. In the multiple piece configuration, the diaphragm cover 32 inserted into the flexible tether loop of the flexible connector 34 may comprise an ascending skirt 44 along its perimeter edge to clip onto the diaphragm of the stethoscope or the flexible loop of the tether may have a gripping edge that may be used to secure the cover to the diaphragm of the stethoscope.

The device of the present invention may be prepared for a variety of stethoscopes including those made by for example, Littmann (3M Company, St. Paul, Minn.), Prestige Medical (Northridge Calif.) MDF Instruments (Westlake Village, Calif.) and Omron Healthcare (Lake Forest, Calif.). They may be made for a variety of stethoscopes including those with interchangeable heads, for Adults, pediatrics and infants with diameter sizes ranging from about 19 mm, about 25 mm, about 33 mm, about 43 mm, about 51 mm, about 54 mm or other size. In some instances these diaphragms may not be circular but more oblong. The present invention may also be prepared in any shape from circular, oval, oblong or egg-shaped.

### Make and Use

In the single piece configuration the device is prepared by vacuum form molding or other similar type technique. If this method is selected it may be best to first cut the piece from a flat sheet of stock material and then place it in a mold to hold it in position before applying the heat and vacuum. A variety of methods known in the art that can be used to create the single piece configuration. In a multiple piece construction the diaphragm cover may be made using the vacuum form molding techniques described above. The flexible tether with a U-shaped bell end on one side may be prepare by injection molding or other similar method used by those skilled in the art to prepare flexible pieces. Once the tether is produced the diaphragm is inserted into the loop end of the tether and held in place by the compression of the loop exerted upon the perimeter edge of the diaphragm or the groove provided on the perimeter edge of the diaphragm inserted into the groove provided along the interior edge of the loop. To further secure the diaphragm in place a flexible adhesive may be used, or the two parts may be fused with heat. Other methods known in the art may be utilized.

During an examination the doctor removes the previous cover on the stethoscope by grasping one or more of the pull tabs provided in the edge of the device. This may be an extension of the ascending side of the one piece configuration or may be one or more pull tabs provided by and integral to the flexible tether holding the diaphragm cover. Once removed the device may be discarded. A new device is applied to the diaphragm of the stethoscope by pressing the diaphragm end of the stethoscope into the ascending skirt of the diaphragm cover until the single piece device snaps over the edges of the diaphragm of the stethoscope. If there is a second end to the device the physician or nurse bends the tether and press fits the second end over the remaining diaphragm or bell end of the dual sided stethoscope. In a multiple piece configuration the diaphragm end of the stethoscope is press fit into the gripping edge of the tether loop holding the diaphragm cover until the tether loop edge has fitted the diaphragm cover evenly over the diaphragm of the stethoscope. If there is a second end to the device the physician or nurse bends the tether and press fits the second end over the remaining diaphragm or bell end of the dual sided stethoscope.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

I claim:

1. A stethoscope cover for an acoustic stethoscope with a dual-sided chest piece having a diaphragm end and a bell end comprising:
a semi-rigid single piece cover having a first end that affixes to said diaphragm end and a second end that affixes to said bell end, wherein said first end and said second end are connected by a tether flap; wherein said first end having a flat surface and a perimeter edge, wherein said perimeter edge having an ascending skirt about a majority of said perimeter edge, wherein said ascending skirt receives said first end securing said flat surface of said first end parallel to said diaphragm of said stethoscope and wherein said second end having a U-shaped circular ring able to receive said second end and encompassing a majority of said second end and securing said U-shaped circular ring to said second end.

2. The stethoscope cover according to claim 1, wherein said cover is disposable.

3. A stethoscope cover for an acoustic stethoscope with a dual-sided chest piece having a first diaphragm end and a second diaphragm end comprising:
a semi-rigid single piece cover having a first end that affixes to said first diaphragm end and a second end that affixes to said second diaphragm end, wherein said first end and said second end are connected by a tether flap, wherein said first end having a first flat surface and a first perimeter edge, wherein said first perimeter edge having a first ascending skirt about a majority of said first perimeter edge, wherein said first ascending skirt receives said first end securing said first flat surface of said first end parallel to said first diaphragm of said stethoscope and wherein said second end having a second flat surface and a second perimeter edge, wherein said second perimeter edge having a second ascending skirt about a majority of said second perimeter edge, wherein said second ascending skirt receives said second end securing said second flat surface of said second end parallel to said second diaphragm of said stethoscope.

4. The stethoscope cover according to claim 3, wherein said cover is disposable.

5. A stethoscope cover for an acoustic stethoscope with a dual-sided chest piece having a first diaphragm end and a second diaphragm end comprising:
a first semi-rigid circular cover for said first diaphragm end;
a second semi-rigid circular cover for said second diaphragm end; and
a flexible connector having a first end to which said first semi-rigid circular cover is affixed and a second end to which said second semi-rigid circular cover is affixed, wherein said first end connects to said first diaphragm end and said second end connects to said second diaphragm end, wherein said first end and said second end are connected by a tether flap, wherein said first end having a perimeter edge, wherein said perimeter edge having an ascending skirt about a majority of said perimeter edge, wherein said ascending skirt receives said first end securing said first semi-rigid circular cover to said first diaphragm end parallel to said first diaphragm of said stethoscope and wherein said second end having a perimeter edge, wherein said perimeter edge having an ascending skirt about a majority of said perimeter edge, wherein said ascending skirt receives said second end securing said second semi-rigid circular cover to said second diaphragm end parallel to said second diaphragm of said stethoscope.

6. A stethoscope cover for an acoustic stethoscope with a dual-sided chest piece having a diaphragm end and a bell end comprising:
a semi-rigid circular cover for said diaphragm end; and
a flexible connector having a first end to which said first semi-rigid circular cover is affixed and a second end, wherein said first end connects to said diaphragm end and said second end connects to said bell end, wherein said first end and said second ends are connected by a tether flap, wherein said first end having a perimeter edge, wherein said perimeter edge having an ascending skirt about a majority of said perimeter edge, wherein said ascending skirt receives said diaphragm end securing said semi-rigid circular cover to said diaphragm end parallel to said diaphragm of said stethoscope and wherein said second end having a U-shaped circular ring able to receive said bell end.

7. A stethoscope cover for an acoustic stethoscope having at least one diaphragm end comprising:
a semi-rigid single piece cover that affixes to said at least one diaphragm end, wherein said single piece having a flat surface and a perimeter edge, wherein said perimeter edge having an ascending skirt that receives said at least one diaphragm end securing said flat surface of said semi-rigid single piece cover parallel to said at least one diaphragm end of said stethoscope.

8. The stethoscope cover of claim 7, further comprising a removal means.

9. The stethoscope cover of claim 8, wherein said removal means is a tab.

10. A stethoscope cover for an acoustic stethoscope having at least one bell end comprising:
a semi-rigid single piece cover that affixes to said bell end, wherein said semi-rigid single piece cover having a U-shaped circular ring able to receive said bell end.

11. The stethoscope cover of claim 10, farther comprising a removal means.

12. The stethoscope cover of claim 11, wherein said removal means is a tab.
